(19) United States
(12) Patent Application Publication
(10) Pub. No.: US 2020/0000574 A1
(43) Pub. Date: Jan. 2, 2020

(54) Bioabsorbable Deformable Anchors

(71) Applicant: Acuitive Technologies, Inc., Allendale, NJ (US)

(72) Inventors: Alex Khowaylo, Naples, FL (US); Michael P. McCarthy, Ho-Ho-Kus, NJ (US); Rui J. Ferreira, Livingston, NJ (US); Peter Krijger, Rockaway, NJ (US); Minh-Tuan Richard Tran, Ho-Ho-Kus, NJ (US); Douglas William Gabel, Pompton Plains, NJ (US)

(73) Assignee: Acuitive Technologies, Inc., Allendale, NJ (US)

(21) Appl. No.: 16/465,384

(22) PCT Filed: Nov. 30, 2017
(86) PCT No.: PCT/US2017/061068

§ 371 Date: May 30, 2019

Related U.S. Application Data
(60) Provisional application No. 62/428,323, filed on Nov. 30, 2016.

(51) Int. Cl. A61F 2/08

(52) U.S. Cl. CPC A61F 2/0811; A61F 2002/0835; A61B 2017/0414; A61F 2002/0852; A61F 2002/0888

(57) ABSTRACT

Biodegradable and bioabsorbable anchors and anchor systems for use in musculoskeletal fixation applications comprising (1) an anchor body comprising a longitudinal axis, a proximal end, a distal end, an outer surface, and a bore extending from the proximal end and parallel to the longitudinal axis, wherein the bore defines an inner surface of the anchor body, and wherein at least a portion of the anchor body is expandable in a direction non-parallel to the longitudinal axis, and (2) an expansion pin comprising a longitudinal axis, a proximal end, a distal end, and a surface, and configured for insertion into the bore such that, when inserted, it expands the expandable portion of the anchor body in a direction non-parallel to the longitudinal axis. Both the disclosed anchors and anchor systems are at least in part formed from a citrate-based polymer.

### CROSS-REFERENCE TO RELATED APPLICATION

The present application claims priority benefit to a US provisional application entitled “Bioabsorbable Deformable Anchors,” which was filed on Nov. 30, 2016, and assigned Ser. No. 62/428,323. The entire content of the foregoing provisional application is incorporated herein by reference.

### TECHNICAL FIELD

The present disclosure relates to biodegradable and bioabsorbable anchors and anchor systems for use in musculoskeletal fixation applications, as well as to methods of using such anchors and anchor systems to repair musculoskeletal tissue.

### BACKGROUND

Current bone anchors generally rely on a fixed shape that either threads into or is press fit into a bone substrate, which exerts stress on the area surrounding the anchor placement. This stress prevents surgeons from placing anchors in close proximity to each other and the bone quality needs to be dense to provide adequate initial fixation. Accordingly, there exists a continuing need for improved anchors and anchor systems for use in musculoskeletal fixation applications that have an enhanced capability for deflection, deformation, or manipulation (e.g., expansion) of the shape of the anchor body, thus creating an enlarged surface area, resulting in a more rigid attachment, and concomitantly, improved fixation. The present disclosure provides for such improved anchors and anchor systems.

### Embodiments of the Invention

One embodiment of the present invention relates to a bioabsorbable anchor system comprising (1) an anchor body comprising a longitudinal axis, a proximal end, a distal end, an outer surface, and a bore extending from the proximal end and parallel to the longitudinal axis, wherein the bore defines an inner surface of the anchor body, and wherein at least a portion of the anchor body is expandable in a direction non-parallel to the longitudinal axis, and (2) an expansion pin comprising a longitudinal axis, a proximal end, a distal end, and a surface, and configured for insertion into the bore such that, when inserted, it expands the expandable portion of the anchor body in a direction non-parallel to the longitudinal axis, wherein the anchor system is at least in part formed from a citrate-based polymer.

In the above embodiment, the outer surface of the anchor body can comprise one or more protrusions extending outwardly therefrom in a direction non-parallel to the longitudinal axis. In certain embodiments, the one or more protrusions can be selected from the group consisting of barbs, knurls, threads, ribs, ridges, tines, teeth, wedges, fins, and any combination thereof. In certain embodiments, the one or more protrusions can extend radially from the longitudinal axis. In certain embodiments, the one or more protrusions can further comprise one or more flexible outer ridges.

In the above embodiment, at least a portion of the expansion pin can have a circumference greater than the circumference of at least a portion of the bore. In certain embodiments, at least a portion of the bore can have a circumference that decreases in size moving from the proximal end of the anchor body towards the distal end of the anchor body. In certain embodiments, at least a portion of the bore can have a circumference that decreases in size moving from the distal end of the anchor body towards the proximal end of the anchor body.

In the above embodiment, the width of at least a portion of the distal end of the anchor body can be greater than the width of at least a portion of the proximal end of the anchor body. In certain embodiments, the width of at least a portion of the proximal end of the anchor body can be greater than the width of at least a portion of the distal end of the anchor body.

In the above embodiment, the bore can extend through the distal end of the anchor body.

In the above embodiment, at least a portion of the distal end of the expansion pin can have a circumference greater than that of at least a portion of the bore at the distal end of the anchor body. In certain embodiments, the distal end of the expansion pin can comprise a substantially spherical tip, wherein at least a portion of the spherical tip has a circumference greater than that of at least a portion of the bore at the distal end of the anchor body. In certain embodiments, the substantially spherical tip further comprises a hole or eyelet configured to accommodate a suture. In certain embodiments, the distal end of the expansion pin can comprise a tapered tip, wherein at least a portion of the tapered tip has a circumference greater than that of at least a portion of the bore at the distal end of the anchor body. In certain embodiments, the tapered tip further comprises a hole or eyelet configured to accommodate a suture.

In the above embodiment, the anchor body can further comprise one or more radial slots extending from the proximal end of the anchor body towards the distal end of the anchor body and/or extending from the distal end of the anchor body towards the proximal end of the anchor body, wherein the one or more slots are parallel to the longitudinal axis. In certain embodiments, the anchor body can comprise one radial slot extending the entire length of the anchor body, wherein the slot is parallel to the longitudinal axis.

In the above embodiment, the surface of the expansion pin can further comprise one or more protrusions extending outwardly therefrom in a direction non-parallel to the longitudinal axis of the expansion pin. In certain embodiments, the one or more protrusions are selected from the group consisting of barbs, knurls, threads, ribs, ridges, tines, teeth, wedges, fins, and any combination thereof. In certain embodiments, the expansion pin is cannulated. In certain embodiments, he cannulation is configured to accommodate a suture.

In the above embodiment, the surface at the proximal end of the expansion pin can further comprise at least one barb that extends radially from the longitudinal axis of the expansion pin and the inner surface of the anchor body can further comprise at least one groove configured such that, when the expansion pin is inserted into the bore, the at least one barb is locked into the at least one groove, thereby preventing proximal and/or distal movement of the expansion pin relative to the anchor body. In certain embodiments, the bore can have a length greater than that of the expansion pin.

In the above embodiment, the anchor body can be cylindrical in shape, the distal end of the anchor body can be conical in shape, the one or more protrusions extending from the outer surface of the anchor body can be barbs or threads that extend radially from the longitudinal axis of the anchor body, a radial slot can extend from the proximal end of the anchor body towards the distal end of the anchor body, and the anchor body and extension pin can be formed from the polycondensation product of citric acid and/or citrate with at least one C₄ to C₁₂ alkane diol. In certain of these embodiments, the distal end of the expansion pin can comprise a substantially spherical tip, wherein at least a portion of the substantially spherical tip has a circumference greater than that of at least a portion of the bore at the distal end of the anchor body, and wherein the substantially spherical tip optionally comprises a hole or eyelet configured to accommodate a suture. In certain other of these embodiments, the distal end of the expansion pin can comprise a tapered tip, wherein at least a portion of the tapered tip has a circumference greater than that of at least a portion of the bore at the distal end of the anchor body and wherein the tapered tip comprises hole or eyelet configured to accommodate a suture.

In the above embodiment, the anchor body can be cylindrical in shape, the distal end of the anchor body can be conical in shape, the one or more protrusions extending from the outer surface of the anchor body can be barbs or threads that extend radially from the longitudinal axis of the anchor body, at least two radial slots can extend from the proximal end of the anchor body towards the distal end of the anchor body and at least two radial slots can extend from the distal end of the anchor body towards the proximal end of the anchor body, and the anchor body and extension pin can be formed from the polycondensation product of citric acid and/or citrate with at least one C₄ to C₁₂ alkane diol.

In the above embodiment, the anchor body can be cylindrical in shape, the distal end of the anchor body can be conical in shape, the one or more protrusions extending from the outer surface of the anchor body can be barbs or threads that extend radially from the longitudinal axis of the anchor body, at least two radial slots can extend from the proximal end of the anchor body towards the distal end of the anchor body or from the distal end of the anchor body towards the proximal end of the anchor body, the proximal end of the expansion pin can comprise at least one barb that extends radially from the longitudinal axis of the expansion pin, the inner surface of the anchor body can comprises at least one groove configured such that, when the expansion pin is inserted into the bore, the at least one barb is locked into the at least one groove, thereby preventing proximal and/or distal movement of the expansion pin relative to the anchor body, and the anchor body and extension pin are formed from the polycondensation product of citric acid and/or citrate with at least one C₄ to C₁₂ alkane diol. In certain of these embodiments, the bore has a length greater than that of the expansion pin.

In the above embodiment, the anchor body is cylindrical in shape, the distal end of the anchor body is conical in shape, the one or more protrusions extending from the outer surface of the anchor body are barbs or threads that extend radially from the longitudinal axis of the anchor body, at least one radial slot extends from the proximal end of the anchor body towards the distal end of the anchor body or from the distal end of the anchor body towards the proximal end of the anchor body, at least a portion of the expansion pin is substantially conical in shape and threaded, at least a portion of the inner surface of the anchor body is threaded such that, when the expansion pin is screwed into the bore, the expansion pin is secured into the anchor body, thereby preventing proximal and/or distal movement of the expansion pin relative to the anchor body, and the anchor body and extension pin are formed from the polycondensation product of citric acid and/or citrate with a least one C₄ to C₁₂ alkane diol.

Another embodiment of the present invention relates to a bioabsorbable anchor comprising a body, wherein the body comprises (1) a longitudinal axis, (2) a proximal end, (3) a distal end, and (4) an outer surface, wherein the outer surface comprises one or more protrusions extending outwardly and radially therefrom in a direction non-parallel to the longitudinal axis, wherein the anchor is at least in part formed from a citrate-based polymer.

In the above embodiment, the one or more protrusions can further comprise one or more flexible outer ridges.

In the above embodiment, the bioabsorbable anchor can further comprise a bore extending transversely through the anchor and non-parallel to the longitudinal axis.

In the above embodiment, the body of the bioabsorbable anchor is cylindrical in shape, the distal end is conical in shape, the one or more protrusions are barbs or threads, and the anchor is formed from the polycondensation product of citric acid and/or citrate with at least one C₄ to C₁₂ alkane diol. In certain embodiments, the bioabsorbable anchor can further comprise a bore extending transversely through the anchor, wherein the bore is located at the proximal end of the anchor and is perpendicular to the longitudinal axis.

In the above embodiments, the anchor body of the above bioabsorbable anchors and anchor systems can comprise a radial cross-sectional geometry selected from the group consisting of circular, ovoid, triangular, quadrangular, pentagonal, and hexagonal. In certain embodiments, the shape of the anchor body is selected from the group consisting of cylindrical, conical, a triangular prism, a quadrangular prism, a pentagonal prism, and a hexagonal prism. In certain embodiments, the distal end of the anchor body is conical in shape.

In the above embodiments, the above bioabsorbable anchors and anchor systems can at least in part be formed from a citrate-based (co)polyester. In certain embodiments, the citrate-based (co)polyester can be the polycondensation product of citric acid and/or citrate with a least one C₄ to C₁₂ alkane diol. In certain embodiments, the citrate-based (co)polyester can be poly(1,8-octanediol citrate). In certain embodiments, the above bioabsorbable anchors and anchor systems can at least in part be formed from a composite comprising a citrate-based polymer and a bioceramic. In certain embodiments, the bioceramic is selected from the group consisting of hydroxyapatite and beta-tricalcium phosphate.

Yet another embodiment of the present invention relates to methods for repairing musculoskeletal tissue. In certain embodiments, the method can comprise (1) identifying or creating a cavity in bone tissue, (2) inserting the anchor body of the above bioabsorbable anchor system into the cavity, and (3) inserting the expansion pin into the bore of the anchor body. In certain other embodiments, the method can comprise (1) identifying or creating a cavity in bone tissue, (2) preloading the expansion pin of the above bioabsorbable anchor system into the anchor body, (3) inserting the preloaded anchor body into the cavity, and (4) proximally tensioning the expansion pin such that the tapered tip is translated into the bore of the anchor body. In certain other embodiments, the method can comprise (1) identifying or creating a cavity in bone tissue, and (2) inserting the above bioabsorbable anchor into the cavity. In certain embodiments, this method can further comprise loading the anchor into a thin-walled inserter having the same general cross-sectional shape as the anchor, wherein the cross-sectional area of the thin-walled inserter is slightly smaller than that of the cavity, inserting the thin-walled inserter preloaded with the anchor into the cavity, and removing the inserter so as to leave behind the anchor in the cavity, wherein (a) the cross-sectional shape of the cavity roughly approximates that of the anchor, (b) the cross-sectional area of the anchor is slightly larger than that of the cavity.

### BRIEF DESCRIPTION OF THE DRAWINGS

The foregoing and other features and advantages provided by the present disclosure will be more fully understood from the following description of exemplary embodiments when read together with the accompanying drawings.

FIG. 1 depicts a front view of the separate components of an exemplary bioabsorbable anchor system according to the present disclosure.

FIG. 2 depicts a front view of the integrated components of an exemplary bioabsorbable anchor system according to the present disclosure.

FIG. 3 depicts an offset view of the integrated components of an exemplary bioabsorbable anchor system according to the present disclosure.

FIG. 4 depicts a cross-sectional view of the integrated components of an exemplary bioabsorbable anchor system according to the present disclosure.

FIG. 5 depicts a front view of the tapered distal tip of an expansion pin (not shown) according to the present disclosure.

FIG. 6 depicts an offset view of the integrated components of an exemplary bioabsorbable anchor system according to the present disclosure.

FIG. 7 depicts a cross-sectional view of the integrated components of an exemplary bioabsorbable anchor system according to the present disclosure.

FIG. 8 depicts an offset view of the separate components of an exemplary bioabsorbable anchor system according to the present disclosure.

FIG. 9 depicts a front view of the separate components of an exemplary bioabsorbable anchor system according to the present disclosure.

FIG. 10 depicts a cross-sectional view of the integrated components of an exemplary bioabsorbable anchor system according to the present disclosure.

FIG. 11 depicts a cross-sectional view of the integrated components of an exemplary bioabsorbable anchor system according to the present disclosure.

FIG. 12 depicts an offset view of an exemplary bioabsorbable anchor according to the present disclosure.

FIG. 13 depicts an offset view of an exemplary bioabsorbable anchor according to the present disclosure.

FIG. 14 depicts a blow-up of flexible outer ridges according to the present disclosure.

FIG. 15 depicts an offset view of an exemplary bioabsorbable anchor according to the present disclosure.

FIG. 16 depicts two cross-sectional views of the integrated components of an exemplary bioabsorbable anchor system according to the present disclosure.

FIG. 17 depicts an offset view of the integrated components of an exemplary bioabsorbable anchor system according to the present disclosure.

FIG. 18 depicts two cross-sectional views of the integrated components of an exemplary bioabsorbable anchor system according to the present disclosure.

FIG. 19 depicts an offset view of the integrated components of an exemplary bioabsorbable anchor system according to the present disclosure.

FIG. 20 depicts steps of an exemplary method of repairing tissue using a bioabsorbable anchor according to the present disclosure.

FIG. 21 depicts a step of an exemplary method of repairing tissue using a bioabsorbable anchor according to the present disclosure.

FIG. 22 depicts a step of an exemplary method of repairing tissue using a bioabsorbable anchor according to the present disclosure.

FIG. 23 depicts a step of an exemplary method of repairing tissue using a bioabsorbable anchor according to the present disclosure.

FIG. 24 depicts an offset view of an exemplary bioabsorbable anchor according to the present disclosure.

FIG. 25 depicts an offset cross-sectional view of integrated components of an exemplary bioabsorbable anchor system according to the present disclosure.

FIG. 26 depicts an offset view of integrated components of an exemplary bioabsorbable anchor system according to the present disclosure.

FIG. 27 depicts an offset cross-sectional view of integrated components of an exemplary bioabsorbable anchor system according to the present disclosure.

FIG. 28 depicts an offset view of integrated components of an exemplary bioabsorbable anchor system according to the present disclosure.

FIG. 29 depicts an offset cross-sectional view of integrated components of an exemplary bioabsorbable anchor system according to the present disclosure.

FIG. 30 depicts an offset view of an exemplary bioabsorbable anchor according to the present disclosure.

FIG. 31 depicts a front view of an exemplary bioabsorbable anchor according to the present disclosure.

### DETAILED DESCRIPTION

In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting. Any ranges described herein will be understood to include the endpoints and all values between the endpoints.

In various aspects, configurations, and embodiments, the present disclosure provides for bioabsorbable anchors and anchor systems manufactured from elastomeric citrate-based polymers that possess features that allow for deflection, deformation, or manipulation (e.g., expansion) of the shape of the anchor body, resulting in a more rigid attachment, and concomitantly improved fixation, of the anchor to the surrounding musculoskeletal tissue. The presently disclosed bioabsorbable anchor systems comprise an anchor body and an expansion pin. The anchor body comprises a longitudinal axis, a proximal end, a distal end, an outer surface, and a bore extending from the proximal end and parallel to the longitudinal axis. The bore defines an inner surface of the anchor body. At least a portion of the anchor body is expandable in a direction non-parallel to the longitudinal axis. The expansion pin comprises a longitudinal axis, a proximal end, a distal end, and a surface. It is configured for insertion into the bore such that, when inserted, it expands the expandable portion of the anchor body in a direction non-parallel to the longitudinal axis. The presently disclosed bioabsorbable anchors comprise a body that comprises a longitudinal axis, a proximal end, a distal end, and an outer surface. The outer surface, in turn, comprises one or more protrusions extending outwardly and radially therefrom in a direction non-parallel to the longitudinal axis and further comprise one or more flexible outer ridges. The presently disclosed bioabsorbable anchor can further include a bore extending transversely through the anchor and non-parallel to the longitudinal axis. Both the presently disclosed bioabsorbable anchors and anchor systems are at least in part manufactured from a citrate-based polymer.

The presently disclosed bioabsorbable anchors and the anchor bodies of the presently disclosed bioabsorbable anchor systems can be of any suitable length and width. In certain embodiments, the width of the distal end of the anchor body of the presently disclosed bioabsorbable anchor systems is greater than the width of the proximal end of the anchor body. In certain other embodiments, the width of the proximal end of the anchor body of the presently disclosed bioabsorbable anchor systems is greater than the width of the distal end of the anchor body.

The presently disclosed bioabsorbable anchors and the anchor bodies of the presently disclosed bioabsorbable anchor systems can be of any suitable shape. Examples of such shapes include, but are not limited to, those that have radial cross-sectional geometry selected from the group consisting of circular, ovoid, triangular, quadrangular, pentagonal, and hexagonal. Further examples of such shapes include, but are not limited to, cylindrical, conical, a triangular prism, a quadrangular prism, a pentagonal prism, and a hexagonal prism. In certain embodiments, the distal ends of the presently disclosed bioabsorbable anchors and the anchor bodies of the presently disclosed bioabsorbable anchor systems are conical in shape so as to facilitate insertion of the anchor or anchor body into a cavity in tissue. In certain embodiments, the distal ends of the presently disclosed bioabsorbable anchors and the anchor bodies of the presently disclosed bioabsorbable anchor systems can comprise a hole or eyelet configured to accommodate a suture.

The presently disclosed bioabsorbable anchors and anchor systems are at least in part manufactured from any suitable citrate-based polymer having the requisite elastomericity to facilitate deflection, deformation, or manipulation (e.g., expansion) of the shape of the anchor or anchor body. In certain embodiments, the citrate-based polymer is a citrate-based (co)polyester (i.e., a homopolyester or copolyester). Examples of such citrate-based (co)polyesters include, but are not limited to, those prepared by the polymerization (i.e., polycondensation) of citric acid and/or citrate with C₂ to C₂₀ alkanediols. In certain embodiments, the citrate-based (co)polyester is the polycondensation product of citric acid and/or citrate with C₄ to C₁₂ alkanediols. In certain embodiments, the citrate-based (co)polyester is poly(1,8-octanediol citrate).

Alternatively, the presently disclosed bioabsorbable anchors and anchor systems can at least in part be manufactured from a suitable composite comprising the above citrate-based polymers and a bioceramic. Examples of such bioceramics include, but are not limited to, hydroxyapatite and beta-tricalcium phosphate. The citrate-based polymer(s) and the bioceramic(s) can be present in the composite in any suitable weight ratio relative to each other. Examples of such weight ratios of citrate-based polymer(s) to bioceramic(s) include, but are not limited to, 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, and 1:99% by weight. In certain embodiments, certain portions of the presently disclosed bioabsorbable anchors and anchor systems can have different weight ratios of citrate-based polymer to bioceramic than other portions. In certain embodiments, certain portions of the presently disclosed bioabsorbable anchors and anchor systems can be manufactured from the above composite(s), while other portions can be manufactured from citrate-based polymer alone.

The bore can extend partially or completely (i.e., from the proximal end through the distal end) through the anchor bodies of the presently disclosed bioabsorbable anchor systems. The bore of the anchor bodies of the presently disclosed bioabsorbable anchor systems can be of any suitable circumference. In certain embodiments, the circumference of the bore can be uniform through the length of the bore. In certain other embodiments, the circumference of all or a portion of the bore may decrease in size moving from the proximal end of the anchor body towards the distal end of the anchor body. In certain other embodiments, the circumference of all or a portion of the bore may decrease in size moving from the distal end of the anchor body towards the proximal end of the anchor body.

The outer surface of the presently disclosed bioabsorbable anchors and the anchor bodies of the presently disclosed bioabsorbable anchor systems can comprise one or more protrusions extending outwardly therefrom in a direction non-parallel to the longitudinal axis (i.e., at an angle greater than 0° and less than 180° relative to the longitudinal axis) that can resist motion in one or several directions. The protrusions can be of any suitable size. In certain embodiments, the protrusions extend from the outer surface perpendicularly relative to the longitudinal axis. In certain other embodiments, the protrusions extend from the outer surface at an angle of 45° or 135° relative to the longitudinal axis. Examples of such protrusions include, but are not limited to, barbs, knurls, threads, ribs, ridges, tines, teeth, wedges, fins, or any combination thereof. The protrusions can extend radially from the longitudinal axis. In other words, a single protrusion may extend from the surface of the anchor body uniformly around the longitudinal axis. Additionally, the protrusions can further comprise one or more flexible outer ridges. The presently disclosed bioabsorbable anchors and the anchor bodies of the presently disclosed bioabsorbable anchor systems can also comprise voids, such as grooves, slots, and holes, and/or porosity in order to aid in deformation of the anchor or anchor body prior to facilitate insertion into the tissue cavity.

The anchor body of the presently disclosed bioabsorbable anchor systems can comprise one or more radial slots extending from the proximal end of the anchor body towards the distal end of the anchor body. Alternatively, or in addition thereto, the anchor body of the presently disclosed bioabsorbable anchor systems can comprise one or more radial slots extending from the distal end of the anchor body towards the proximal end of the anchor body. The slots are parallel to the longitudinal axis. In certain embodiments, the anchor body comprises one radial slot extending the entire length of the anchor body. In certain embodiments, the anchor body comprises one, two, three, four, five, six, seven, or eight radial slots extending the partial length of the anchor body from the proximal and/or the distal end of the anchor body.

The expansion pins of the presently disclosed bioabsorbable anchor systems can have any suitable length and circumference. In certain embodiments, the expansion pin is longer in length than the bore. In certain embodiments, the expansion pin is the same length as the bore. In certain embodiments, the expansion pin is shorter in length than the bore. At least a portion of the expansion pin must have a circumference greater than the circumference of at least a portion of the bore, such that, when the expansion pin is completely inserted into the bore, expansion of at least a portion of the anchor body is achieved. In certain embodiments, the distal end of the expansion pin has a circumference greater than that of the bore at the distal end of the anchor body. In certain embodiments, the proximal end of the expansion pin has a circumference greater than that of the bore at the proximal end of the anchor body. In certain embodiments, the expansion pin is cannulated. In other words, the expansion pin can itself comprise a bore that extends completely (i.e., from the proximal end through the distal end) through the expansion pin. In certain of these embodiments, this cannulation is configured to accommodate a suture. Alternatively, in certain embodiments, the a suture can molded into the expansion pin. In certain of those embodiments, the molded suture can be configured (e.g., protrude from the proximal end of the expansion pin) to provide a means for pulling the pin proximally so as to cause expansion of the distal end of the anchor body against the tissue (i.e., in embodiments where the distal end or tip of the expansion pin have a larger circumference than that of the bore.

The distal end of the expansion pin can further comprise a tip, at least a portion of which has a circumference greater than that of the bore at the distal end of the anchor body. In certain embodiments, the tip is substantially spherical in shape. In certain other embodiments, the tip is tapered in shape. The expansion pin tip, whether substantially spherical or tapered in shape, can further comprise a hole or eyelet configured to accommodate attachment of a suture and to aid in suture placement, tightening, and/or retention.

The surface of the expansion pin can further comprise at least one protrusion that extends outwardly therefrom in a direction non-parallel to the longitudinal axis of the expansion pin (i.e., at an angle greater than 0° and less than 180° relative to the longitudinal axis) and that can resist motion in one or several directions. The protrusion(s) can be of any suitable size. In certain embodiments, the protrusion(s) extend from the surface perpendicularly relative to the longitudinal axis. In certain other embodiments, the protrusion(s) extend from the outer surface at an angle of 45° or 135° relative to the longitudinal axis of the expansion pin. Examples of such protrusion(s) include, but are not limited to, barbs, knurls, threads, ribs, ridges, tines, teeth, wedges, fins, or any combination thereof. The protrusion(s) can extend radially from the longitudinal axis of the expansion pin. In other words, a single protrusion may extend from the surface of the expansion pin uniformly around its longitudinal axis. The protrusion(s) can be located anywhere along the length of the expansion pin. In certain embodiments, the protrusion(s) are located at the proximal end of the expansion pin. In certain embodiments, the protrusion(s) are located at the distal end of the expansion pin.

In certain embodiments, the protrusion(s) are barb(s). In conjunction with this feature, the inner surface of the anchor body further comprises groove(s) configured such that, when the expansion pin is inserted into the bore, the barb(s) are locked into the groove(s), thereby preventing proximal and/or distal movement of the expansion pin relative to the anchor body. The size and spacing of the barb(s) and groove(s) can be varied to control the amount and range of expansion. In certain embodiments, these barb(s) and groove(s) can be located at the respective proximal or distal ends of the anchor body and expansion pin. In certain other embodiments, the protrusion(s) are threads and at least a portion of the expansion pin is substantially conical in shape. In conjunction with this feature, the inner surface of the anchor body further comprises threads configured such that, when the expansion pin is screwed into the bore, the anchor body is expanded while the expansion pin is simultaneously secured in the bore, thereby preventing proximal and/or distal movement of the expansion pin relative to the anchor body. In certain embodiments, these threads can be located at the respective proximal or distal ends of the anchor body and expansion pin. In these threaded embodiments, the proximal end of the expansion pin can have any suitable drive head that enables the expansion pin to be screwed into the anchor body).

The presently disclosed bioabsorbable anchors and anchor systems can be used to repair musculoskeletal tissue. Examples of such repairs include, but are not limited to, anchoring any type of soft fixation to hard tissue, such as anchoring sutures attached to tendons or ligaments to bone (e.g., use as tendinosis anchors), approximating two or more bone sections to each other, such as a fractured clavicle, or to close a sternotomy (e.g., use as interference screws), or to fixate a plate to anatomy where screws would traditionally be used. In this last example, the anchor or anchor body would further comprise a head, so that the plate would be secured against the tissue. An example of a method of repairing tissue using the present disclosed bioabsorbable anchor system includes, but is not limited to, the steps of (1) identifying or creating a cavity in bone tissue, (2) inserting the anchor body of the presently disclosed bioabsorbable anchor system into the cavity, and (3) inserting the expansion pin into the bore of the anchor body. Alternatively, prior to insertion into the cavity, the anchor body can be preloaded with an expansion pin having a tip with a circumference greater than that of the bore at the distal end of the anchor body. The preloaded anchor body is then inserted into the cavity and tensioned in the proximal direction such that the tip is translated into the bore of the anchor body. An example of a method of repairing tissue using the present disclosed bioabsorbable anchors includes, but is not limited to, the steps of (1) identifying or creating a cavity in bone tissue, and (2) inserting the anchor into the cavity.

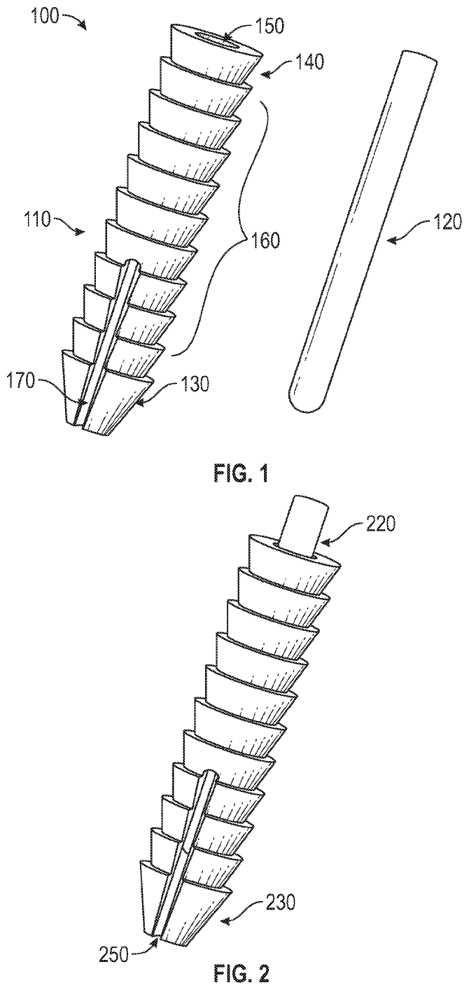

Examples of the bioabsorbable anchors and anchor systems according to the present disclosure are illustrated in FIGS. 1-31.

With reference to FIG. 1, shown therein is an exemplary embodiment of the presently disclosed bioabsorbable anchor system 100 comprising an anchor body 110 and an expansion pin 120. Anchor body 110 has a distal end 130 of greater width than proximal end 140 and a bore 150 that extends from the proximal end 140 through the distal end 130. Anchor body 110 also comprises a plurality of radially extending barbs 160 and a single radial slot 170 that allows the distal end 130 to compress. In one embodiment, this exemplary embodiment can be used in the following manner. First, a hole having a smaller width than the distal end 130 of the anchor body 110 is drilled into an implantation site and anchor body 110 is then press-fitted into the cavity, causing distal end 130 to compress radially such that the circumference of bore 150 at distal end 130 is smaller than that of the expansion pin 120. Once anchor body 110 is implanted to a pre-determined depth, expansion pin 120 is then inserted down the length of bore 150, as shown in FIG. 2. As expansion pin 220 approaches distal end 230, force is exerted against bore 250, forcing distal end 230 to expand radially, compressing it against the surrounding tissue and resulting in a more rigid attachment.

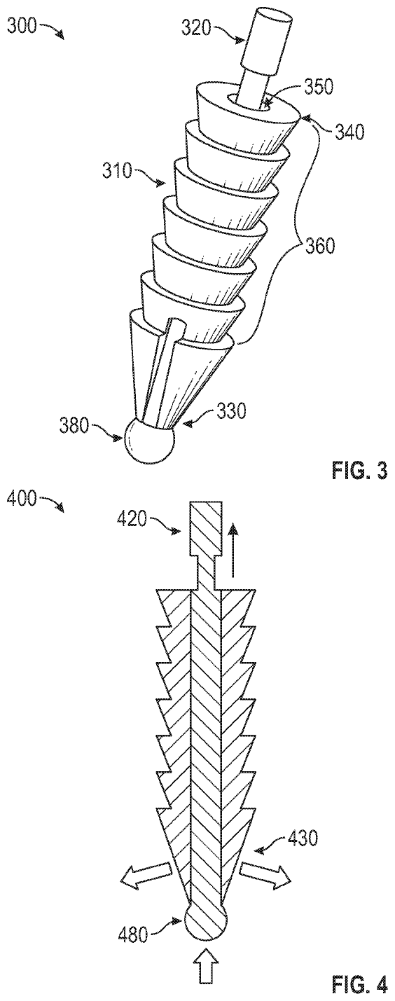

With reference to FIG. 3, shown therein is an exemplary embodiment of the presently disclosed bioabsorbable anchor system 300 comprising an anchor body 310 preloaded with an expansion pin 320. Anchor body 310 has a distal end 330, a proximal end 340, and a bore 350 that extends from the proximal end 340 through the distal end 330. Anchor body 310 also comprises a plurality of radially extending barbs 360. Expansion pin 320 comprises a spherical distal tip 380 having a circumference greater than that of bore 350. Once inserted into a cavity at the implantation site, expansion pin 420 of the preloaded system 400 is tensioned proximally, causing spherical distal tip 480 to translate into distal end 430, as shown in FIG. 4. This causes distal end 430 to expand radially, compressing it against the surrounding tissue and resulting in a more rigid attachment.

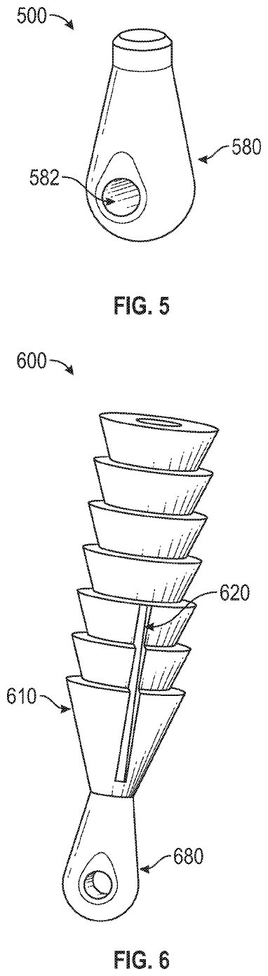

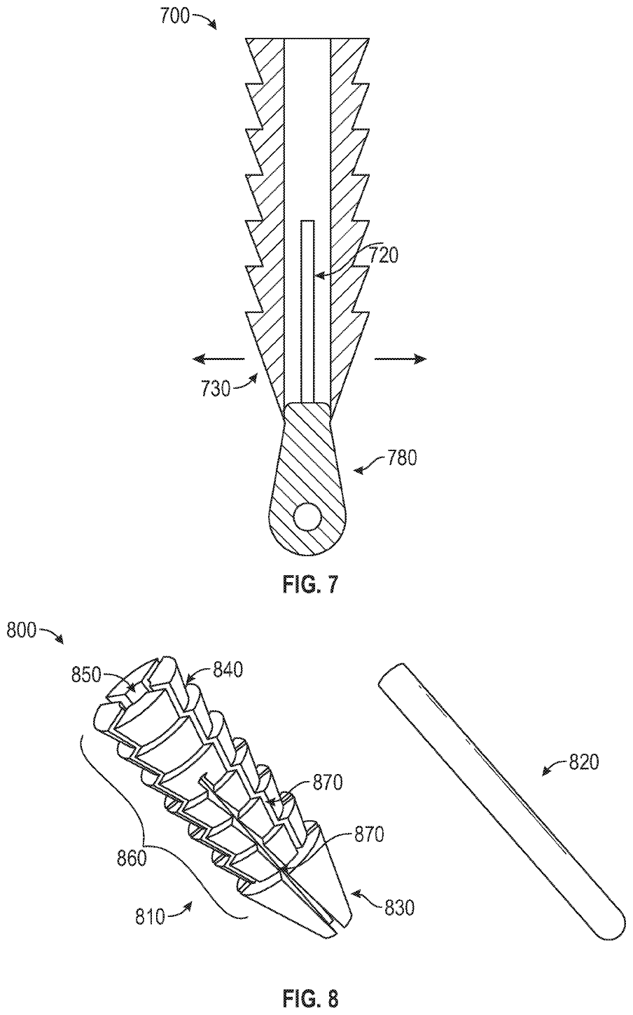

With reference to FIG. 5, shown therein is an alternative exemplary embodiment of a distal tip having a circumference greater than that of the bore. Alternative embodiment 500 comprises a tapered distal tip 580 of an expansion pin (not shown) having an eyelet 582 capable of accommodating attachment of a suture. FIG. 6 illustrates an exemplary embodiment of the presently disclosed bioabsorbable anchor system 600 comprising an anchor body 610 preloaded with an expansion pin 620 comprising such a distal tip 680. As with the spherical distal tip embodiment illustrated in FIGS. 3 and 4, once preloaded system 700 is inserted into a cavity at the implantation site, expansion pin 720 is tensioned proximally, causing tapered distal tip 780 to translate into distal end 730, as shown in FIG. 7. This causes distal end 730 to expand radially, compressing it against the surrounding tissue and resulting in a more rigid attachment.

With reference to FIG. 8, shown therein is an exemplary embodiment of the presently disclosed bioabsorbable anchor system 800 comprising an anchor body 810 and an expansion pin 820. Anchor body 810 has a distal end 830, a proximal end 840, and a bore 850 that extends from the proximal end 840 through the distal end 830. Anchor body 810 also comprises a plurality of radially extending barbs 860 and a plurality of radial slots 870 located at both the distal end 830 and the proximal end 840 that allows anchor body 810 to compress upon implantation, such that the circumference of bore 850 is smaller than that of expansion pin 820. Insertion of expansion pin 820 into bore 850 causes radial expansion along the entire length of anchor body 810.

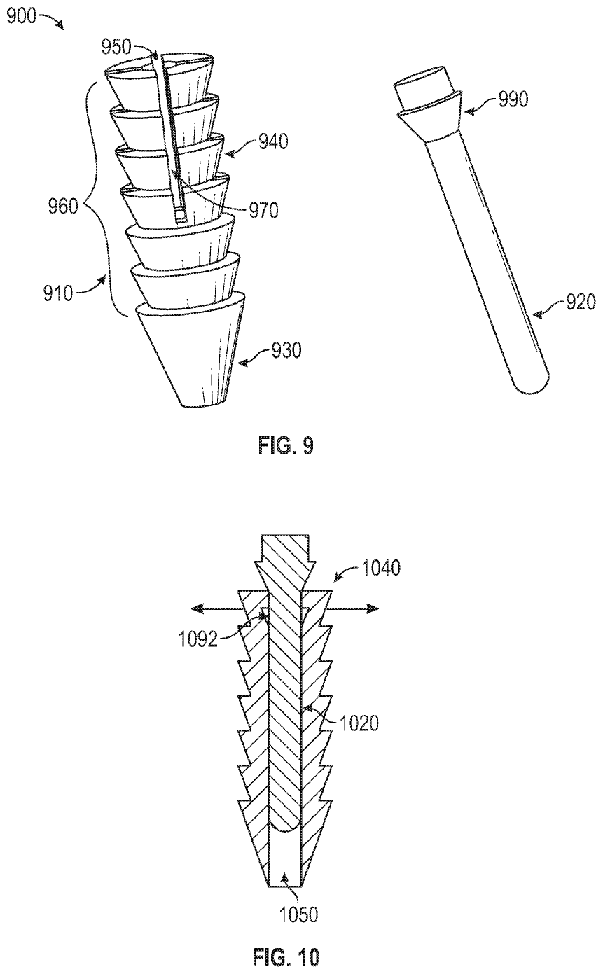

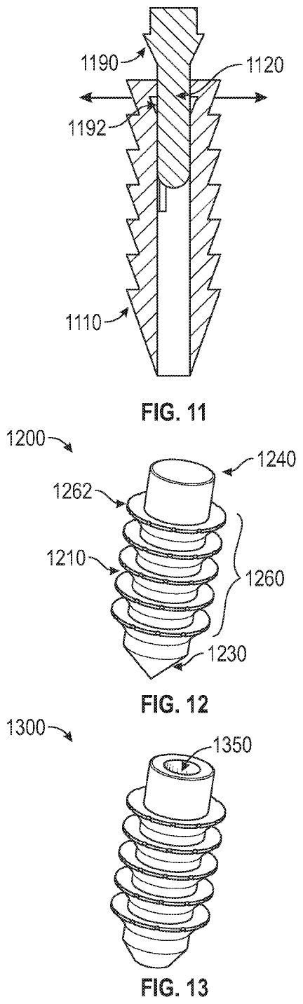

With reference to FIG. 9, shown therein is an exemplary embodiment of the presently disclosed bioabsorbable anchor system 900 comprising an anchor body 910 and an expansion pin 920. Anchor body 910 has a distal end 930, a proximal end 940, and a bore 950 that extends from the proximal end 940 through the distal end 930. Anchor body 910 also comprises a plurality of radially extending barbs 960 and a plurality of radial slots 970 located at the proximal end 940 that allows anchor body 910 to expand upon insertion of expansion pin 920. Expansion pin 920 comprises a radially extending barb 990 at its proximal end. As shown in FIG. 10, bore 1050 further comprises a groove 1092 located towards proximal end 940. Insertion of expansion pin 1020 causes proximal end 1040 to flare outward, compressing it against the surrounding tissue and resulting in a more rigid attachment. Groove 1092 is configured such that, when the expansion pin 1020 is inserted into bore 1050, barb 1090 is locked into groove 1092, thereby preventing proximal movement of expansion pin 1020 relative to the anchor body 1010. As shown in FIG. 11, expansion pin 1120 can be shorter than anchor body 1110, since only barb 1190 engages anchor body 1110 at groove 1192 for expansion. Expansion pin length and the number and shape of the radial slots can be modulated to vary the degree and shape of expansion.

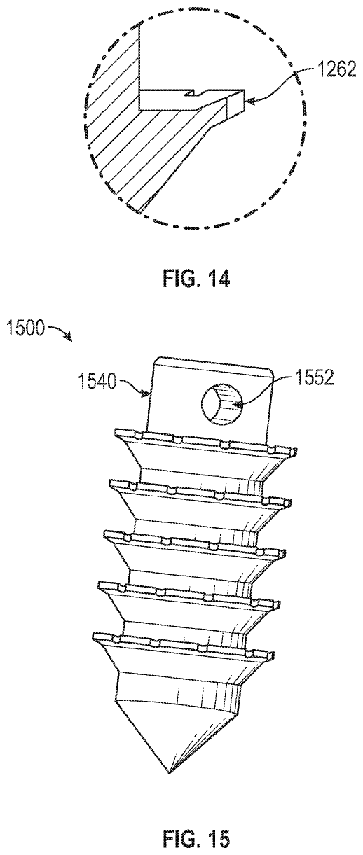

With reference to FIG. 12, shown therein is an exemplary embodiment of the presently disclosed bioabsorbable anchor 1200 comprising a body 1210 having a distal end 1230, a proximal end 1240, a plurality of radially extending barbs 1260, each of which further comprise a plurality of flexible outer ridges 1262 (a blow-up of these flexible outer ridges is illustrated in FIG. 14). If anchor 1200 experiences force proximally (e.g., pulled on in the direction opposite the way it was inserted), flexible outer ridges 1262 flex outward and expand their grip on against the adjacent tissue. FIG. 13 illustrates an alternative exemplary embodiment of this bioabsorbable anchor, whereby the anchor 1300 also comprises a bore 1350 capable of accommodating an expansion pin having a circumference greater than that of the bore. FIG. 15 illustrates an alternative exemplary embodiment of this bioabsorbable anchor, whereby anchor 1500 also comprises a bore 1552 extending transversely through the proximal end 1540 of anchor 1500.

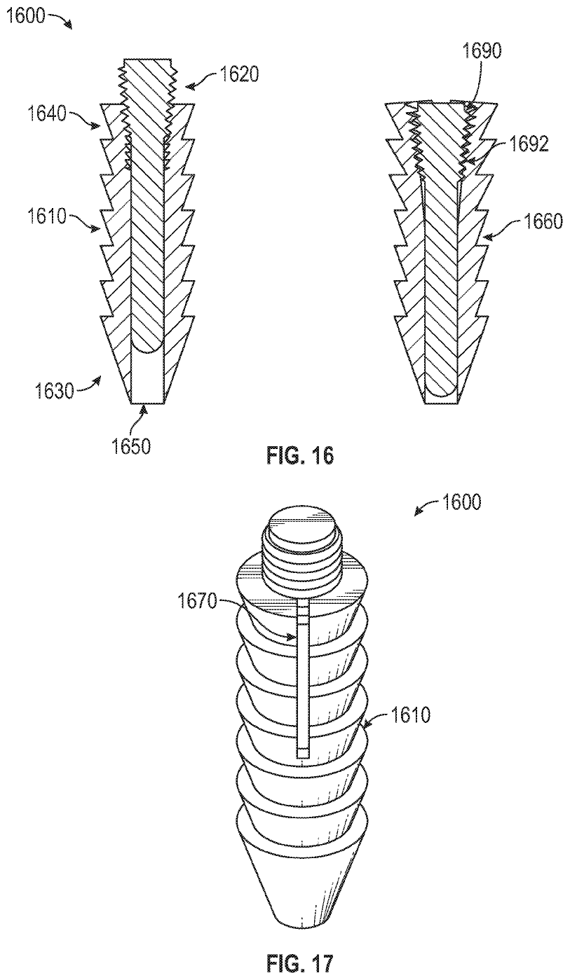

With reference to FIGS. 16 and 17, shown therein is an exemplary embodiment of the presently disclosed bioabsorbable anchor system 1600 comprising an anchor body 1610 and an expansion pin 1620. Anchor body 1610 has a distal end 1630, a proximal end 1640, and a bore 1650 that extends from the proximal end 1640 through the distal end 1630. Anchor body 1610 also comprises a plurality of radially extending barbs 1660 and a radial slot 1670 located at the proximal end 1640 that allows anchor body 1610 to expand upon insertion of expansion pin 1620. Expansion pin 1620 comprises a substantially conical, threaded proximal end 1690. Bore 1650 further comprises threads 1692 on the interior surface of proximal end 1640. Insertion of expansion pin 1620 causes proximal end 1640 to flare outward, compressing it against the surrounding tissue and resulting in a more rigid attachment. Substantially conical, threaded proximal end 1690 and 1692 is configured such that, when the expansion pin 1620 is screwed into bore 1650, anchor body 1610 and expansion pin 1620 are secured together, thereby preventing proximal movement of expansion pin 1620 relative to the anchor body 1610.

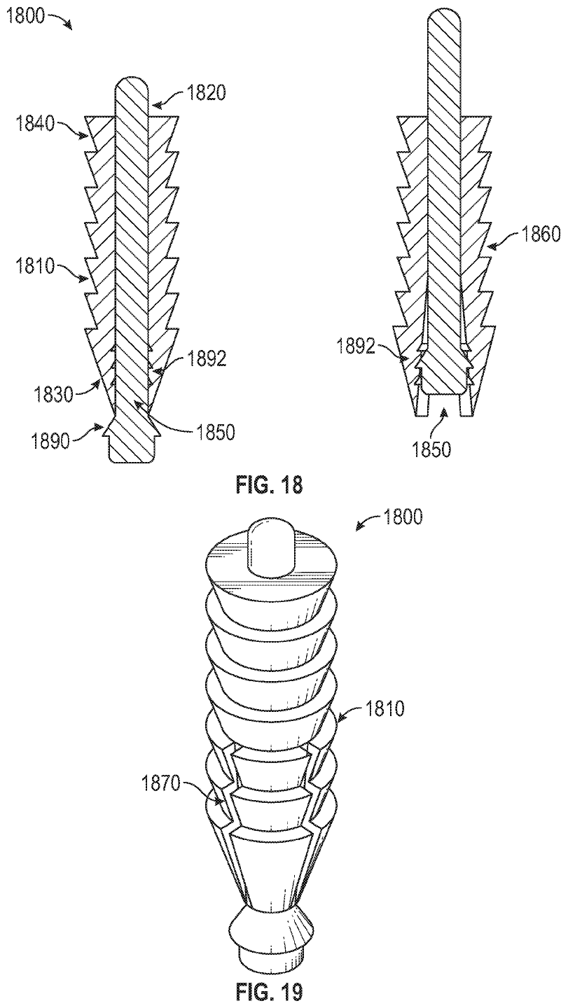

With reference to FIGS. 18 and 19, shown therein is an exemplary embodiment of the presently disclosed bioabsorbable anchor system 1800 comprising an anchor body 1810 and an expansion pin 1820. Anchor body 1810 has a distal end 1830, a proximal end 1840, and a bore 1850 that extends from the proximal end 1840 through the distal end 1830. Anchor body 1810 also comprises a plurality of radially extending barbs 1860 and a plurality of radial slots 1870 located at the distal end 1840 that allows anchor body 1810 to expand upon insertion of expansion pin 1820. Expansion pin 1820 comprises a radially extending barb 1890 at its distal end. Bore 1850 further comprises a plurality of grooves 1892 located toward distal end 1830. Insertion of expansion pin 1820 causes proximal end 1840 to flare outward, compressing it against the surrounding tissue and resulting in a more rigid attachment. Grooves 1892 are configured such that, when the expansion pin 1820 is inserted into bore 1850, barb 1890 is locked into the first of grooves 1892, thereby preventing proximal movement of expansion pin 1820 relative to the anchor body 1810. As expansion pin 1820 is advanced into further into the bore 1850 and locked into subsequent grooves 1892, the anchor body 1810 can be expanded against the adjacent tissue proximally.

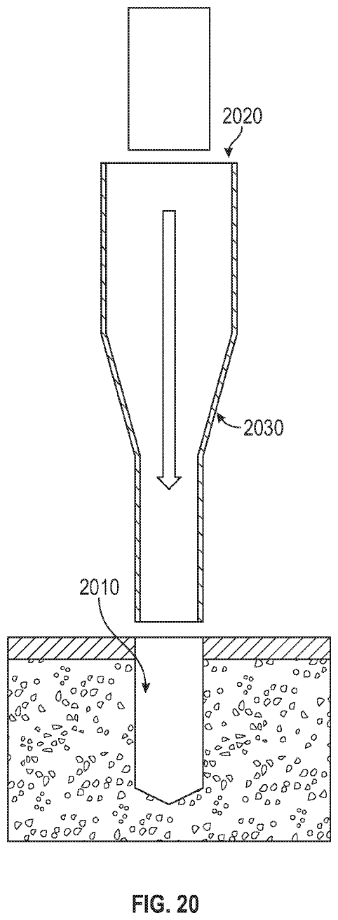

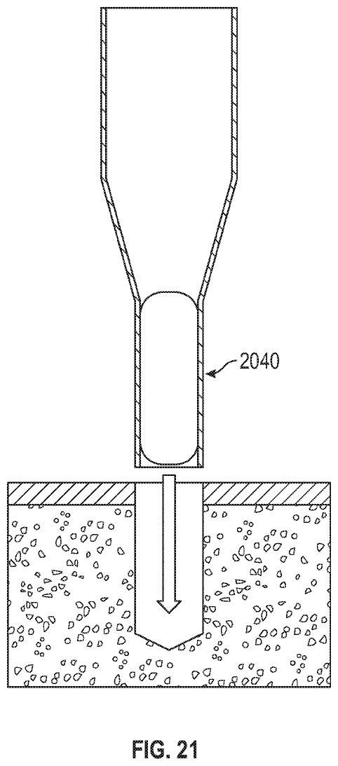

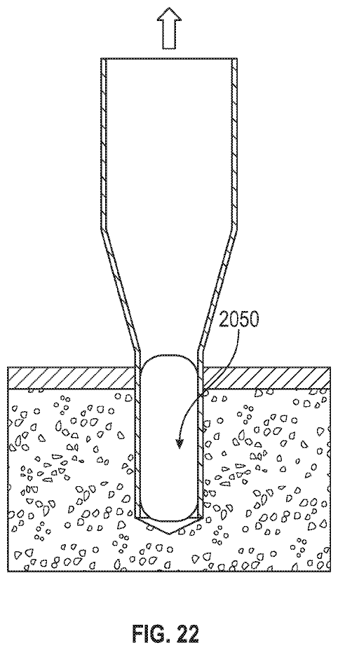

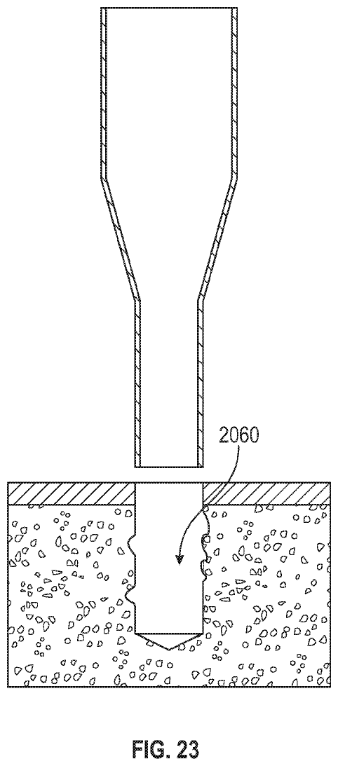

With reference to FIGS. 20-23, shown therein is an exemplary method of repairing tissue using the presently disclosed bioabsorbable anchors. As shown in FIG. 20, this exemplary method begins with the steps of 2010 creating a cavity in bone tissue, the cross-sectional shape of which roughly approximates that of the anchor, wherein the cross-sectional area of the anchor is slightly larger than that of the cavity and 2020 loading the anchor into a thin-walled inserter having the same general cross-sectional shape as the anchor, wherein the cross-sectional area of the thin-walled inserter is slightly smaller than that of the cavity. In certain embodiments, and as shown in FIG. 20, the barrel of the thin-walled inserter can be tapered 2030 in shape so as to facilitate the gradual squeezing the anchor to a cross-sectional area smaller than that of the cavity. As shown in FIG. 21, the thin-walled inserter preloaded with the anchor 2040 is inserted into the cavity. In certain embodiments, the anchor can comprise a hole or eyelet configured to accommodate a suture. Once the preloaded inserter is inserted into the cavity, any sutures present may be adjusted at this stage. As shown in FIG. 22, the inserter is then removed to leave behind the anchor 2050. Upon removal of the thin-walled inserter, the anchor then expands into the cavity and beyond into the interstices of the prepared bone 2060, as shown in FIG. 23.

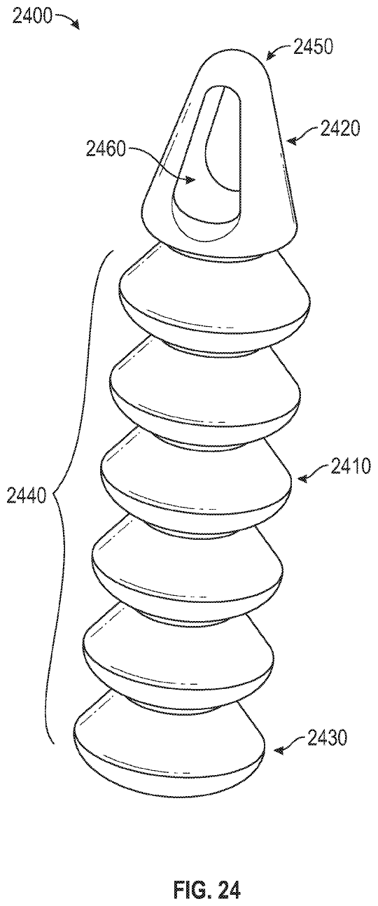

With reference to FIG. 24, shown therein is an exemplary embodiment of the presently disclosed bioabsorbable anchor 2400 comprising a citrate-based polymer body 2410 having a distal end 2420, a proximal end 2430, a plurality of radially extending barbs 2440, and a substantially conical tip 2450 comprising an eyelet 2460, wherein the substantially conical tip has a higher ceramic content than the remainder of the body of the anchor. This exemplary embodiment is capable being used in the method illustrated in FIGS. 20-23. The more elastic polymer body facilitates compression within the thin-walled inserter, but then returns to its original size and expands into the interstices of the prepared bone cavity upon removal of the inserter. The higher ceramic content eyelet tip assembles with and retains sutures during clinical loading.

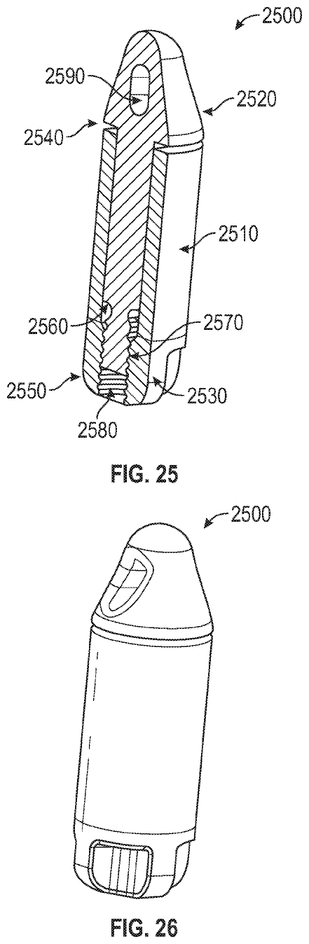

With reference to FIGS. 25 and 26, shown therein is an exemplary embodiment of the presently disclosed bioabsorbable anchor system 2500 comprising an anchor body 2510, a proximal plate 2520, and a distal plate 2530. Anchor body 2510 has a proximal end 2540, a distal end 2550, and a bore 2560 that extends from the proximal end 2540 through the distal end 2550. Anchor body 2510 has an initial outside diameter substantially similar to the diameter of the cavity in the bone tissue. Proximal plate 2520, i.e., an expansion pin, comprises a threaded distal end 2570 and a substantially conical head with a hole 2590 in a direction non-parallel to the longitudinal axis. Distal plate 2530 comprises threads 2580 on the interior surface.

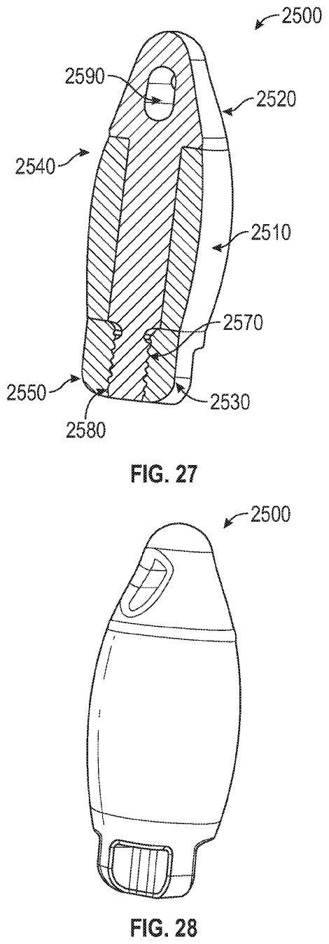

Insertion of proximal plate 2520, through anchor body 2510, to interface with distal plate 2530, causes anchor body 2510 to deform outwardly in a semi-circular shape, increasing its diameter (as depicted in FIGS. 27 and 28), and compressing it against the surrounding tissue and resulting in a more rigid attachment. Threaded distal end 2570 and 2580 is configured such that, when the proximal plate 2520 is inserted into bore 2550 of anchor body 2510 and screwed into distal plate 2530, anchor body 2510 is rigidly secured between proximal plate 2520 and distal plate 2530. Anchor body 2510 is at least in part formed from a citrate-based polymer. The outer surface of anchor body 2510 can be smooth, as depicted, or can include at least one protrusion, extending outwardly and radially therefrom in a direction non-parallel to the longitudinal axis. The hole 2590, located through the substantially conical head of the proximal plate 2520, can engage with a suture (not depicted). In another embodiment, a loop can be affixed to the distal plate 2530 to secure a suture or soft tissue thereto. In another embodiment, a cam mechanism can compress the proximal plate 2520 and the distal plate 2530 together to constrain the anchor body 2510 from movement.

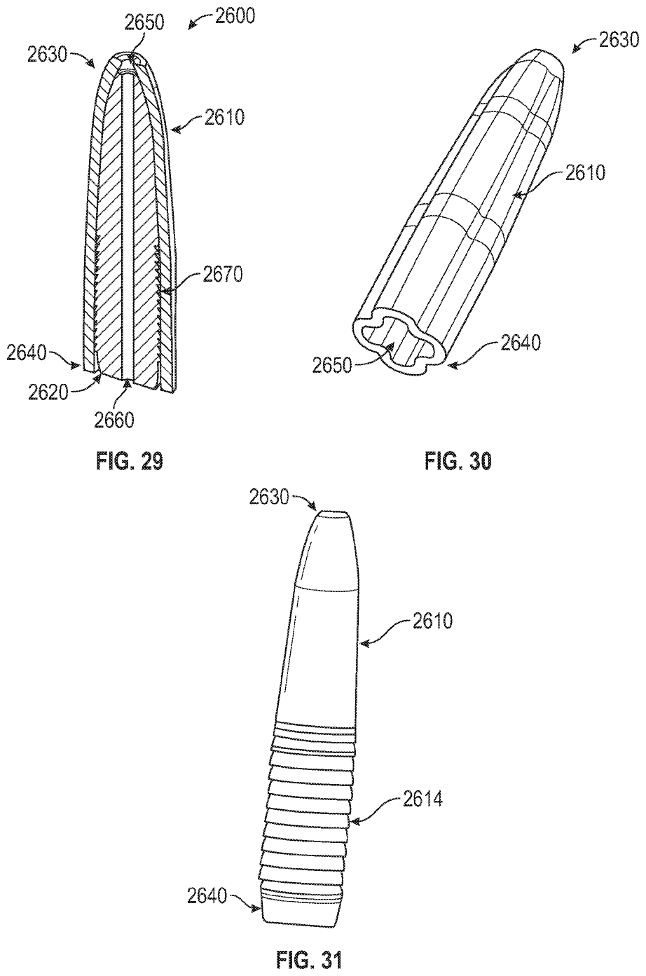

With reference to FIG. 29 in view of FIG. 30, shown therein is an exemplary embodiment of the presently disclosed bioabsorbable anchor system 2600 comprising an anchor body 2610 and one or more expansion pins 2620. Anchor body 2610 has a proximal end 2630, a distal end 2640, and a bore 2650 that is longitudinally located. Anchor body 2610 is substantially conical in shape to receive one or more expansion pins 2620. Anchor body 2610 is generally flexible so as to partially reshape when the less flexible expansion pin 2620 is fully inserted. Expansion pin 2620 has the same general conical shape as anchor body 2610, but tapers distally 2640 to allow for a gradual transition from full interference to no interference. Expansion pin 2620 can comprise of one or more protrusions 2670 extending outwardly and radially therefrom in a direction non-parallel to the longitudinal axis. Expansion pin 2620 further comprises a bore 2660 that extends from the proximal end 2630 through the distal end 2640.

Insertion of expansion pin 2620 into anchor body 2610 reshapes the anchor body 2610, thereby compressing it against the surrounding tissue and resulting in a more rigid attachment. The protrusions 2670 located on the outer surface of expansion pin 2620 further secure the expansion pin 2620 to the bore 2650 of the anchor body 2610. Although depicted as one expansion pin 2620, one or more expansion pins 2620 can be inserted in anchor body 2610 at various longitudinal locations and for differing insertion depths. Anchor body 2610 is at least in part formed from a citrate-based polymer. The outer surface of anchor body 2610 can be smooth, as depicted in FIGS. 29 and 30, or can comprise of one or more protrusions 2614 extending outwardly and radially therefrom in a direction non-parallel to the longitudinal axis, as depicted in FIG. 31.

The above bioabsorbable anchors and anchor systems, with specific reference to FIGS. 25-31, can comprise a radial cross-sectional geometry selected from the group consisting of circular, ovoid, triangular, quadrangular, pentagonal, and hexagonal. In certain embodiments, the shape of the anchor body is selected from the group consisting of cylindrical, conical, a triangular prism, a quadrangular prism, a pentagonal prism, and a hexagonal prism.

Although the inventive concepts disclosed and claimed herein and the advantages thereof have been described in detail, it should be understood that various changes, substitutions and alterations can be made herein without departing from the spirit and scope thereof as defined by the appended claims. Moreover, the scope of the present application is not intended to be limited to the particular embodiments of the process, apparatus, items of manufacture, compositions of matter, means, methods, and steps described in the specification. As one of ordinary skill in the art will readily appreciate from the disclosure of the presently disclosed and claimed inventive concepts, various processes, apparatus, items of manufacture, compositions of matter, means, methods, or steps, presently existing or later to be developed that perform substantially the same function or achieve substantially the same result as the corresponding embodiments described herein may be utilized according to the presently disclosed and claimed inventive concepts. Accordingly, the appended claims are intended to include within their scope such processes, apparatus, items of manufacture, compositions of matter, means, methods, or steps.

1. A bioabsorbable anchor system comprising:
an anchor body comprising a longitudinal axis, a proximal end, a distal end, an outer surface, and a bore extending from the proximal end and parallel to the longitudinal axis, wherein the bore defines an inner surface of the anchor body, and wherein at least a portion of the anchor body is expandable in a direction non-parallel to the longitudinal axis; and
an expansion pin comprising a longitudinal axis, a proximal end, a distal end, and a surface, and configured for insertion into the bore such that, when inserted, it expands the expandable portion of the anchor body in a direction non-parallel to the longitudinal axis;
wherein the anchor system is at least in part formed from a citrate-based polymer.

2. The bioabsorbable anchor system of claim 1, wherein the outer surface of the anchor body comprises one or more protrusions extending outwardly therefrom in a direction non-parallel to the longitudinal axis of the anchor body.

3. The bioabsorbable anchor system of claim 2, wherein the one or more protrusions are selected from the group consisting of barbs, knurls, threads, ribs, ridges, tines, teeth, wedges, fins, and any combination thereof.

4. (canceled)

5. The bioabsorbable anchor system of claim 2, wherein the one or more protrusions further comprise one or more flexible outer ridges.

6. The bioabsorbable anchor system of claim 1, wherein at least a portion of the expansion pin has a circumference greater than the circumference of at least a portion of the bore.

7. (canceled)

8. (canceled)

9. (canceled)

10. (canceled)

11. The bioabsorbable anchor system of claim 1, wherein the bore extends through the distal end of the anchor body.

12. The bioabsorbable anchor system of claim 1, wherein at least a portion of the distal end of the expansion pin has a circumference greater than that of at least a portion of the bore at the distal end of the anchor body.

13. The bioabsorbable anchor system of claim 12, wherein the distal end of the expansion pin comprises a substantially spherical tip, wherein at least a portion of the substantially spherical tip has a circumference greater than that of at least a portion of the bore at the distal end of the anchor body.

14. The bioabsorbable anchor system of claim 13, wherein the substantially spherical tip further comprises a hole or eyelet configured to accommodate a suture.

15. (canceled)

16. (canceled)

17. The bioabsorbable anchor system of claim 1, wherein the anchor body further comprises one or more radial slots extending from the proximal end of the anchor body towards the distal end of the anchor body and/or extending from the distal end of the anchor body towards the proximal end of the anchor body, wherein the one or more slots are parallel to the longitudinal axis.

18. (canceled)

19. The bioabsorbable anchor system of claim 1, wherein the surface of the expansion pin further comprises one or more protrusions extending outwardly therefrom in a direction non-parallel to the longitudinal axis of the expansion pin.

20. The bioabsorbable anchor system of claim 19, wherein the one or more protrusions are selected from the group consisting of barbs, knurls, threads, ribs, ridges, tines, teeth, wedges, fins, and any combination thereof.

21. The bioabsorbable anchor system of claim 1, wherein the expansion pin is cannulated.

22. (canceled)

23. The bioabsorbable anchor system of claim 1, wherein the surface of the expansion pin further comprises at least one barb that extends radially from the longitudinal axis of the expansion pin and the inner surface of the anchor body further comprises at least one groove configured such that, when the expansion pin is inserted into the bore, the at least one barb is locked into the at least one groove, thereby preventing proximal and/or distal movement of the expansion pin relative to the anchor body.

24. (canceled)

25. The bioabsorbable anchor system of claim 1, wherein the anchor body comprises a radial cross-sectional geometry selected from the group consisting of circular, ovoid, triangular, quadrangular, pentagonal, and hexagonal.

26. The bioabsorbable anchor system of claim 1, wherein the shape of the anchor body is selected from the group consisting of cylindrical, conical, a triangular prism, a quadrangular prism, a pentagonal prism, and a hexagonal prism.

27. (canceled)

28. The bioabsorbable anchor system of claim 1, wherein the anchor system is at least in part formed from a citrate-based (co)polyester.

29. The bioabsorbable anchor system of claim 28, wherein the citrate-based (co)polyester is the polycondensation product of citric acid and/or citrate with a least one C₄ to C₁₂ alkane diol.

30. The bioabsorbable anchor system of claim 29, wherein the citrate-based (co)polyester is poly(1,8-octanediol citrate).

31. The bioabsorbable anchor system of claim 1, wherein the anchor system is at least in part formed from a composite comprising a citrate-based polymer and a bioceramic.

32. The bioabsorbable anchor system of claim 1, wherein the bioceramic is selected from the group consisting of hydroxyapatite and beta-tricalcium phosphate.

33. The bioabsorbable anchor system of claim 2, wherein:
(a) the anchor body is cylindrical in shape;
(b) the distal end of the anchor body is conical in shape;
(c) the one or more protrusions extending from the outer surface of the anchor body are barbs or threads that extend radially from the longitudinal axis of the anchor body;
(d) a radial slot extends from the proximal end of the anchor body towards the distal end of the anchor body; and
(e) the anchor body and extension pin are formed from the polycondensation product of citric acid and/or citrate with a least one C₄ to C₁₂ alkane diol.

34. The bioabsorbable anchor system of claim 33, wherein
(a) the distal end of the expansion pin comprises a substantially spherical tip, wherein at least a portion of the substantially spherical tip has a circumference greater than that of at least a portion of the bore at the distal end of the anchor body, and wherein the substantially spherical tip optionally comprises a hole or eyelet configured to accommodate a suture.

35. The bioabsorbable anchor system of claim 33, wherein
(a) the distal end of the expansion pin comprises a tapered tip, wherein at least a portion of the tapered tip has a circumference greater than that of at least a portion of the bore at the distal end of the anchor body and wherein the tapered tip comprises a hole or eyelet configured to accommodate a suture.

36. (canceled)

37. (canceled)

38. (canceled)

39. (canceled)

40. The bioabsorbable anchor system of claim 1, further comprising a distal plate for assembly with the expansion pin and the anchor body.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method for repairing musculo skeletal tissue comprising:
identifying or creating a cavity in bone tissue;
inserting the anchor body of the bioabsorbable anchor system of claim 1 into the cavity; and
inserting the expansion pin into the bore of the anchor body.

64. A method for repairing musculo skeletal tissue comprising:
identifying or creating a cavity in bone tissue;
preloading the expansion pin of the anchor system of claim 15 into the anchor body;
inserting the preloaded anchor body into the cavity; and
proximally tensioning the expansion pin such that the tapered tip is translated into the bore of the anchor body.

65. (canceled)

66. (canceled)
